(19) United States
(12) Patent Application Publication
(10) Pub. No.: US 2007/0225258 A1
(43) Pub. Date: Sep. 27, 2007

(54) COMPOSITIONS COMPRISING BISPHOSPHONATE AND AN ANTIFOLATE

(75) Inventor: Michael J. Walsh, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: PROPRIUS PHARMACEUTICALS, INC., San Diego, CA (US)

(21) Appl. No.: 11/670,674

(22) Filed: Feb. 2, 2007

Related U.S. Application Data
(60) Provisional application No. 60/771,174, filed on Feb. 6, 2006.

(51) Int. Cl. A61K 31/675; A61K 31/525

(52) U.S. Cl. 514/89; 514/102; 514/251

(57) ABSTRACT

Compositions and methods for the treatment of arthritis, particularly rheumatoid arthritis and osteoarthritis. These compositions include at least one antifolate and at least one bisphosphonate, or pharmaceutically acceptable salts thereof

### RELATED APPLICATIONS

This application claims priority under 35 U.S.C. 119(e) to U.S. Provisional Patent Application No. 60/771,174, the entire contents of which are incorporated herein by reference.

### FIELD OF THE INVENTION

The present invention relates to compositions comprising at least one bisphosphonate and at least one antifolate. More specifically, the invention relates to the use of these compositions for the treatment of arthritis, including osteoarthritis and rheumatoid arthritis.

### BACKGROUND OF THE INVENTION

Arthritis (‘arth’ meaning joint, ‘itis’ meaning inflammation) actually consists of more than 100 different conditions which can range from relatively mild forms of tendonitis and bursitis, to osteoarthritis (“wear and tear” arthritis), to crippling systemic forms, such as rheumatoid arthritis. Also included within the term “arthritis” are pain syndromes like fibromyalgia and arthritis-related disorders, such as lupus, that involve every part of the body. In addition, gout is also considered a type of arthritis since it affects the joints.

The major forms of arthritis are osteoarthritis and rheumatoid arthritis (RA). Osteoarthritis is the result of long-standing wear and tear on the joints that usually develops as one grows older. It involves a loss of cartilage and a change in bone constitution. In contrast, rheumatoid arthritis (RA) is a chronic inflammatory disorder with systemic features and joint involvement that results in an erosive synovitis, cartilage degradation and joint destruction. Structural damage to the joints is predictive of long-term outcome and contributes to functional decline, disability and the need for major surgery. RA affects more than 2.1 million Americans, 1.5 million of whom are women. This progressive, chronic, and often crippling disease usually starts in middle age but may also occur in children and young adults

Current treatments for RA are focused on treating symptoms (e.g. joint pain, stiffness and swelling) and the underlying disease process. Treatments for RA symptoms include corticosteroids (e.g., prednisone) and nonsteroidal anti-inflammatory drugs (NSAIDs) (e.g., aspirin, ibuprofen, indomethacin, naproxen). Compounds called disease-modifying antirheumatic drugs (DMARDs) (e.g., methotrexate, azothiothioprine, hydroxychloroquine, cyclosporine, D-penicillamine, sulfasalazine, leflunomide and minocycline) and genetically engineered monoclonal antibody-based drugs (e.g., infliximab, etanercept, adalimumab) are targeted to causative factors of RA. The antibody-based drugs target and neutralize an inflammation-causing protein called tumor necrosis factor-α (TNF-α). For the past 20 years, the DMARD of choice has been methotrexate (Rheumatrex), originally developed for the treatment of various cancers. Unfortunately, it is effective in only one in three patients with RA.

Bisphosphonates are carbon-substituted pyrophosphate analogs that have become the treatment of choice for inhibiting excessive osteoclast activity which is a feature of several bone diseases, including osteoporosis and Paget's Disease. These compounds may also have beneficial effects in RA (Curr. Opin. Rheumatol. 15:469-475, 2003; U.S. Pat. No. 5,428,181).

Antifolates, or folate antagonists, are a group of compounds frequently used for cancer treatment. These compounds inhibit thymidylate synthase and dihydrofolate reductase, and reduce de novo purine synthesis. One antifolate, methotrexate, is also used for treatment of rheumatoid arthritis.

Current RA treatments all have their disadvantages and side effects, and not all treatments are effective in all individuals. Thus, there is a need for additional compositions which can effectively treat RA.

### SUMMARY OF THE INVENTION

The present invention provides a method for treating arthritis, particularly RA and osteoarthritis, comprising identifying a mammal in need of such treatment; and administering a bisphosphonate and an antifolate, or a pharmaceutically acceptable salt thereof, to the mammal. In one embodiment, the antifolate is methotrexate In one embodiment, the mammal is a human. In another embodiment, the bisphosphonate is isalendronate, clodronate, etidronate, pamidronate, tiludronate, ibandronate, zolendronate, olpadronate, residronate, neridronate, a substituted derivative thereof, or a pharmaceutically acceptable salt thereof In another embodiment, the antifolate and bisphosphonate are administered simultaneously. In yet another embodiment, the antifolate is administered prior to the bisphosphonate. In still another embodiment, the bisphosphonate is administered prior to the antifolate. The bisphosphonate and antifolate may be administered orally, intravenously, intramuscularly, intra-articularly or rectally.

### DETAILED DESCRIPTION OF THE PREFERRED EMBODIMENT

The present invention relates to the use of one or more bisphosphonates in combination with one or more antifolates, for inhibition of inflammation associated with arthritis, particularly rheumatoid arthritis (RA) and osteoarthritis, and for treatment of these disorders. In the bisphosphonate/antifolate compositions and methods described herein, it will be understood that pharmaceutically acceptable salts, metabolites, prodrugs or substituted derivatives (e.g., esters, amides) of the one or more bisphosphonates and/or antifolate may be used in place of the one or more bisphosphonates and/or antifolate.

Bisphosphonates suitable for use in the present invention include alendronate, clodronate, etidronate, pamidronate, tiludronate, ibandronate, zolendronate, olpadronate, residronate, neridronate, substituted derivatives thereof, and pharmaceutically acceptable salts thereof Other bisphosphonates suitable for us in the present invention include those described in U.S. Pat. Nos. 5,885,473; 6,162,929; 4,705,651; 5,312,954; 4,327,039; 5,196,409; 5,412,141; 4,922,007; 5,019,651; 5,583,122; 6,080,779; and 6,117,856.

Antifolates suitable for use in the present invention include methotrexate, aminopterin, trimetrexate, lometrexol, pemetrexed, 5-fluorouracil and leucovorin. In one embodiment, the antifolate is methotrexate.

Although rheumatoid arthritis and osteoarthritis are the most common arthritic conditions, the compositions of the present invention may also be used to treat other arthritic disorders including Achilles tendonitis, achondroplasia, acromegalic arthropathy, adhesive capsulitis, adult onset Still's disease, ankylosing spondylitis, anserine bursitis, avascular necrosis, Behcet's syndrome, bicipital tendonitis, Blount's disease, brucellar spondylitis, bursitis, calcaneal bursitis, crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, fibromyalgia, Fifth's disease, flat feet, foreign body synovitis, Freiberg's disease, fungal arthritis, Gaucher's disease, giant cell arthritis, gonococcal arthritis, Goodpasture's syndrome, gout, granulomatous arteritis, hemarthrosis, hemochromatosis, henoch-Schonlein purpura, hepatitis B surface antigen disease, hip dysplasia, Hurler syndrome, hypermobility syndrome, hypersensitivity vasculitis, hypertrophic osteoarthopathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epithelial dysplasia, mycoplasmal arthritis, myofascial pain syndrome, neonatal lupus, neuropathic arthropathy, nodular panniculitis, ochronosis, olecranon bursitis, Osgood-Schlatter's disease, osteochondromatosis, osteogenesis imperfecta, osteomalacia, osteomyelitis, osteonecrosis, osteoporosis, overlap syndrome, Paget's disease, palindromic rheumatism, patellofemoral pain syndrome, Pellegrini-Stieda syndrome, pigmented villonodular synovitis, piriformis syndrome, plantar fasciitis, polyarthritis nodosa, polymyalgia rheumatica, polymyositis, popliteal cysts, posterior tibial tendonitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendonitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphyisis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, tietse's syndrome, transient osteoporosis, traumatic arthritis, trochanteric bursitis, tuberculosis arthritis, arthritis of ulcerative colitis, undifferentiated connective tissue syndrome (UCTS), urticarial vasculitis, viral arthritis, Wegener's granulomatosis, Whipple's disease, Wilson's disease and Yersinial arthritis.

The combination of one or more antifolates and one or more bisphosphonates may be used to treat a variety of vertebrates such as birds and mammals. Mammals suitable for treatment using the compositions and methods described herein include humans, primates, dogs, cats, rabbits, guinea pigs, horses, pigs, cows, and the like. A mammal having arthritis (e.g., osteoarthritis or RA) is identified, followed by administration of a pharmaceutical composition comprising one or more antifolates and one or more bisphosphonates, a substituted derivative thereof or a pharmaceutically acceptable salt thereof

The term “pharmaceutical composition” refers to a mixture of one or more bisphosphonates and methotrexate, substituted derivatives thereof or pharmaceutically acceptable salts thereof, with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glutamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which a bisphosphonate and/or methotrexate is converted within the cells of a mammal. The pharmaceutical compositions of the present invention may include a metabolite of a bisphosphonate and/or methotrexate instead of bisphosphonate and/or methotrexate. The scope of the methods of the present invention includes those instances where a bisphosphonate and/or methotrexate is administered to the patient, yet the metabolite is the bioactive entity.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In a further aspect, the present invention relates to a method of treating a patient with a pharmaceutical composition as described herein.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

In the present invention, the antifolate and bisphosphonate may be administered simultaneously; the antifolate may be administered before the bisphosphonate, or vice versa. Suitable routes of administration may, for example, include topical, oral, rectal, transmucosal, or intestinal administration; parenteral delivery, including intramuscular, subcutaneous, intravenous, intramedullary injections, as well as intrathecal, direct intraventricular, intraperitoneal, intranasal, intra-articular (directly into the joint), or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g. by means of conventional mixing, dissolving, granulating, dragee-making, levigating, emulsifying, encapsulating, entrapping or tabletting processes. In the pharmaceutical compositions described herein, the methotrexate and one or more bisphosphonates may be formulated together in the same composition or may be formulated separately. For example, the methotrexate may be provided in a first tablet, and the bisphosphonate(s) may be provided in a separate tablet. If more than one bisphosphonate is included in the composition, they may also be formulated together or separately.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g. in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

For topical administration, the compounds may be formulated for administration to the epidermis as ointments, gels, creams, pastes, salves, gels, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally, including sublingually, which include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g. dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g. gelatin for use in an inhaler or insulator may be formulated containing a powder mix of the compound and a suitable powder base such as lactose or starch.

The compounds may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredients may be in powder form for constitution with a suitable vehicle, e.g. sterile pyrogen-free water, before use.

The compounds may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g. Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage of each component may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 6000 mg of each ingredient, preferably between 1 mg and 5000 mg, e.g. 25 to 5000 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2500 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

### EXAMPLE 1

### Double-Blind Placebo-Controlled Parallel Randomized Study to Evaluate the Efficacy and Safety of Methotrexate Combined with Alendronate (Fosamax)

Inclusion Criteria:

Men and women age 18 to 75 years
Meet American College of Rheumatology (ACR) criteria for RA.
Disease duration of at least 6 months.
Disease onset at >16 years of age.
Must be currently treated with a stable, well-tolerated dose of MTX (7.5 to 20 mg) given once weekly for at least 12 weeks before the baseline visit
Women of childbearing potential, who have a negative pregnancy test result, as well as all male subjects, must agree to use a medically acceptable method of birth control during the study and for at least 12 weeks after the last dose of test article.
Be able and willing to comply with study visits and procedures specified in this protocol.
Understand, sign, and date the written voluntary informed consent form at the screening visit before any protocol-specific procedures are performed.
   Exclusion Criteria:
Any prior use of anti-TNF alpha biologics, rituximab, receipt of anti-CD4 or diphtheria interleukin-2 fusion protein or other immunosuppressive biologics (except for anakinra).
Pregnant or breastfeeding women or women planning to become pregnant during the study or within 12 weeks after the last dose of test article.
History of poor compliance or history of drug abuse/alcohol abuse, excessive alcohol beverage consumption or current or past psychiatric disease that might interfere with the ability to comply with the study protocol or give informed consent.
Any condition that the physician judges could be detrimental to subjects participating in this study, including any clinically important deviations from normal clinical laboratory values or important concurrent medical events, as detailed in the protocol body.

100 patients currently taking methotrexate are administered 10 mg alendronate orally once daily for 60 days. 100 patients are administered a placebo. Alendronate is taken in the morning with a full glass of water at least 30 min before food, beverages or other medications. Treatment efficacy is determined by various measurements, including patient reports of joint stiffness, joint pain, anti-CCP antibody levels, levels of IgM rheumatoid factor and C-reactive protein levels. Patients administered the combination of alendronate and methotrexate show significantly greater improvement than do patients administered placebo or methotrexate alone.

It will be understood by those of skill in the art that numerous and various modifications can be made without departing from the spirit of the present invention. Therefore, it should be clearly understood that the forms of the present invention are illustrative only and are not intended to limit the scope of the present invention.

All documents and other information sources cited above are hereby incorporated in their entirety by reference.

What is claimed is:

1. A method for treating arthritis, comprising identifying a mammal in need of such treatment; and administering a pharmaceutical composition comprising at least one antifolate and at least one bisphosphonate, a substituted derivate thereof, or a pharmaceutically acceptable salt thereof to said mammal.

2. The method of claim 1, wherein said mammal is a human.

3. The method of claim 1, wherein said antifolate is methotrexate.

4. The method of claim 1, wherein said bisphosphonate is selected from the group consisting of alendronate, clodronate, etidronate, pamidronate, tiludronate, ibandronate, zolendronate, olpadronate, residronate, neridronate, substituted derivatives thereof, and pharmaceutically acceptable salts thereof

5. The method of claim 1, wherein said antifolate and said bisphosphonate are administered simultaneously.

6. The method of claim 1, wherein said antifolate is administered prior to said bisphosphonate.

7. The method of claim 1, wherein said bisphosphonate is administered prior to said antifolate.

8. The method of claim 1, wherein said antifolate and said bisphosphonate are in the same formulation.

9. The method of claim 1, wherein said antifolate and said bisphosphonate are in different formulations.

10. The method of claim 1, wherein said antifolate and said bisphosphonate are administered orally, intravenously or intramuscularly.

11. The method of claim 1, wherein said arthritis is rheumatoid arthritis or osteoarthritis.
